(19) United States
(12) Patent Application Publication
(10) Pub. No.: US 2008/0160632 A1
(43) Pub. Date: Jul. 3, 2008

(54) Use of mesoscale self-assembly and recognition to effect delivery of sensing reagent for arrayed sensors

(76) Inventors: C. Grant Willson, Austin, TX (US); Michael Pishko, State College, PA (US); David M. Johnson, Bulverse, TX (US); Ben Rathsack, Richardson, TX (US); Zachary Hogan, Austin, TX (US)

Correspondence Address: Eric B. Meyertons;CONLEY, ROSE & TAYON, P.C., P.O. Box 398, Austin, TX, 78767-0398, US

(21) Appl. No.: 11/981,485

(22) Filed: Oct. 31, 2007

Related U.S. Application Data
(63) Continuation of application No. 10/068,559, filed on Feb. 5, 2002.
(60) Provisional application No. 60/266,824, filed on Feb. 5, 2001.

(51) Int. Cl. G01N 21/62; G01N 33/00

(52) U.S. Cl. 436/171; 422/50; 422/68.1

(57) ABSTRACT

A system and method for the detection of analytes in a fluid, in one embodiment, includes a light source, a sensor array, sensing elements, and a detector. More particularly, the system and method relate to discriminating mixtures of analytes in a fluid. The sensor array is formed from a supporting member into which a plurality of sensing elements may be formed. The sensing element may have a predefined shape. The sensing element may be configured to produce a signal when the sensing element interacts with the analyte. In one embodiment, the identity of the analyte may be determined by the detection of the signal and the shape of the sensing element. Using pattern recognition techniques, the analytes within a multi-analyte fluid may be characterized.

This application claims priority to Provisional Patent Application No. 60/266,824 entitled “The Use of Mesoscale Self-Assembly and Recognition to Effect Delivery of Sensing Reagent for Arrayed Sensors” filed on Feb. 5, 2001.

### BACKGROUND OF THE INVENTION

1. Field of the Invention

The present invention relates to a method and device for the detection of analytes in a fluid. More particularly, the invention relates to the development of a sensor array system capable of discriminating mixtures of analytes in a fluid.

2. Brief Description of the Related Art

The development of smart sensors capable of discriminating different analytes, toxins, and bacteria has become increasingly important for clinical, environmental, health and safety, remote sensing, military, food/beverage and chemical processing applications. Although many sensors capable of high sensitivity and high selectivity detection have been fashioned for single analyte detection, only in a few selected cases have array sensors been prepared which display solution phase multi-analyte detection capabilities. The advantages of such array systems are their utility for the analysis of multiple analytes and their ability to be “trained” to respond to new stimuli. Such on site adaptive analysis capabilities afforded by the array structures make their utilization promising for a variety of future applications. Array based sensors displaying the capacity to sense and identify complex vapors have been demonstrated recently using a number of distinct transduction schemes.

For example, functional sensors based on Surface Acoustic Wave (SAW), tin oxide (SnO₂) sensors, conductive organic polymers, and carbon black/polymer composites have been fashioned. The use of tin oxide sensors, for example, is described in U.S. Pat. No. 5,654,497 to Hoffheins et al. These sensors display the capacity to identify and discriminate between a variety of organic vapors by virtue of small site-to-site differences in response characteristics. Pattern recognition of the overall fingerprint response for the array serves as the basis for an olfaction-like detection of the vapor phase analyte species. Indeed, several commercial “electronic noses” have been developed recently. Most of the well established sensing elements are based on SnO₂ arrays which have been derivatized so as to yield chemically distinct response properties. Arrays based on SAW crystals yield extremely sensitive responses to vapor, however, engineering challenges have prevented the creation of large SAW arrays having multiple sensor sites. To our knowledge, the largest SAW device reported to date possesses only 12 sensor elements. Additionally, limited chemical diversity and the lack of understanding of the molecular features of such systems makes their expansion into more complex analysis difficult.

Other structures have been developed that are capable of identifying and discriminating volatile organic molecules. One structure involves a series of conductive polymer layers deposited onto metal contacting layers. When these sensors are exposed to volatile reagents, some of the volatile reagents adsorb into the polymer layers, leading to small changes in the electrical resistance of these layers. It is the small differences in the behavior of the various sites that allows for a discrimination, identification, and quantification of the vapors. The detection process takes only a few seconds, and sensitivities of part-per-billion can be achieved with this relatively simple approach. This “electronic nose” system is described in U.S. Pat. No. 5,698,089 to Lewis et al. which is incorporated by reference as if set forth herein.

Although the above described electronic nose provides an impressive capability for monitoring volatile reagents, the system possesses a number of undesirable characteristics that warrant the development of alternative sensor array systems. For example, the electronic nose can be used only for the identification of volatile reagents. For many environmental, military, medical, and commercial applications, the identification and quantification of analytes present in liquid or solid-phase samples is necessary. Moreover, the electronic nose systems are expensive (e.g., the Aromascan system costs about $50,000/unit) and bulky (≧1 ft³). Furthermore, the functional elements for the currently available electronic nose are composed of conductive polymer systems which possess little chemical selectivity for many of the analytes which are of interest to the military and civilian communities.

Similar to the electronic nose, array sensors that have shown great analytical promise are those based on the “DNA on a chip” technology. These devices possess a high density of DNA hybridization sites that are affixed in a two-dimensional pattern on a planar substrate. To generate nucleotide sequence information, a pattern is created from unknown DNA fragments binding to various hybridization sites. Both radiochemical and optical methods have provided excellent detection limits for analysis of limited quantities of DNA. (Stimpson, D. I.; Hoijer, J. V.; Hsieh, W.; Jou, C.; Gardon, J.; Theriault, T.; Gamble, R.; Baldeschwieler, J. D. Proc. Natl. Acad. Sci. USA 1995, 92, 6379). Although quite promising for the detection of DNA fragments, these arrays are generally not designed for non-DNA molecules, and accordingly show very little sensitivity to smaller organic molecules. Many of the target molecules of interest to civilian and military communities, however, do not possess DNA components. Thus, the need for a flexible, non-DNA based sensor is still desired. Moreover, while a number of prototype DNA chips containing up to a few thousand different nucleic acid probes have been described, the existing technologies tend to be difficult to expand to a practical size. As a result, DNA chips may be prohibitively expensive for practical uses.

A system of analyzing fluid samples using an array formed of heterogeneous, semi-selective thin films which function as sensing receptor units is described in U.S. Pat. No. 5,512,490 to Walt et al., which is incorporated by reference as if set forth herein. Walt appears to describe the use of covalently attached polymeric “cones” which are grown via photopolymerization onto the distal face of fiber optic bundles. These sensor probes appear to be designed with the goal of obtaining unique, continuous, and reproducible responses from small localized regions of dye-doped polymer. The polymer appears to serve as a solid support for indicator molecules that provide information about test solutions through changes in optical properties. These polymer supported sensors have been used for the detection of analytes such as pH, metals, and specific biological entities. Methods for manufacturing large numbers of reproducible sensors, however, has yet to be developed. Moreover, no methods for acquisitions of data streams in a simultaneous manner are commercially available with this system. Optical alignment issues may also be problematic for these systems.

All of these systems require the placement of the receptors at predetermined locations. The presence or absence of an analyte may be discerned by monitoring a specific location of a sensor array of receptors. Preparing a sensor array with a plurality of receptors at predefined locations may involve complex and expensive processing steps. It is therefore desirable to develop a sensor array system which may be easily manufactured.

### SUMMARY OF THE INVENTION

Herein we describe a system and method for the analysis of a fluid containing one or more analytes. The system may be used for either liquid or gaseous fluids. The system, in some embodiments, may generate patterns that are diagnostic for both the individual analytes and mixtures of the analytes. The system in some embodiments, is made of a plurality of different sensing elements disposed within a supporting member. Each of the different sensing elements may have a shape and/or size that differs from the shape and/or size of the other sensing elements. The shape and/or size of the sensing element may be associated with a specific analyte. Thus, the presence of a particular analyte may be determined by the observance of a signal from a sensing element having a predetermined shape and/or size. This offers an advantage over conventional systems, where the shape and/or size of the particle, rather than the location of the particle, determines the identity of the analyte.

In an embodiment of a system for detecting analytes, the system may include a light source, a sensor, and a detector. The sensor, in some embodiments, is formed of a supporting member which is configured to immobilize the sensing elements. The sensing elements may be arbitrarily distributed throughout the sensor. Alternatively, the sensing elements may be distributed in an ordered array. The sensing elements are configured to create a detectable signal in the presence of an analyte. The sensing elements may produce optical (e.g., absorbance or reflectance) or fluorescence/phosphorescent signals upon exposure to an analyte. The sensing elements may be formed from a polymeric material coupled to a receptor for the analyte. A detector (e.g., a charge-coupled device “CCD”) may be positioned below the sensor to allow for data acquisition. In another embodiment, the detector may be positioned above the sensor to allow for data acquisition from reflectance of the light off of the sensing elements.

Light originating from the light source may pass through the sensor and out through the bottom side of the sensor. Light modulated by the sensing elements may pass through the sensor and onto the proximally spaced detector. Evaluation of the optical changes may be completed by visual inspection or by use of a CCD detector by itself or in combination with an optical microscope. A microprocessor may be coupled to the CCD detector or the microscope.

The sensing elements may include a receptor molecule coupled to a polymeric material. The receptors may interact with one or more analytes. This interaction may take the form of a binding/association of the receptors with the analytes. The supporting member may be made of any material capable of supporting the sensing elements.

A high sensitivity CCD array may be used to measure changes in optical characteristics which occur upon binding of the analytes. The CCD arrays may be interfaced with filters, light sources, fluid delivery and micromachined particle receptacles, so as to create a functional analyte detection system. Data acquisition and handling may be performed with existing CCD technology. CCD detectors may be configured to measure white light, ultraviolet light or fluorescence. Other detectors such as photomultiplier tubes, charge induction devices, photo diodes, photodiode arrays, and microchannel plates may also be used.

A sensing element, in some embodiments, possess both the ability to bind the analyte of interest and to create a modulated signal. The sensing element may include receptor molecules which posses the ability to bind the analyte of interest and to create a modulated signal. Alternatively, the sensing elements may include receptor molecules and indicators. The receptor molecule may posses the ability to bind to an analyte of interest. Upon binding the analyte of interest, the receptor molecule may cause the indicator molecule to produce a signal. The receptor molecules may be naturally occurring or synthetic receptors formed by rational design or combinatorial methods.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 depicts a schematic of an analyte detection system;

FIG. 2 depicts a schematic of a method of producing sensing elements by contact lithography;

FIG. 3 depicts an alternate view of a schematic of a method of producing sensing elements by contact lithography;

FIG. 4 depicts a schematic of a method of producing sensing elements by projection lithography;

FIG. 5 depicts a schematic of a method of producing sensing elements by micromolding;

FIG. 6 depicts a schematic of a method of producing sensing elements by an alternate micromolding technique;

FIG. 7 depicts sensing elements disposed within a support member;

FIG. 8A-B depict a schematic view sensing elements arranged in a predetermined pattern within a support member;

FIG. 9A-C depict a schematic of a method for forming a plurality of sensor from elongated sensing elements;

FIG. 10 depicts a plurality of elongated sensing elements having different shapes disposed within a support material;

FIG. 11 depicts a method of forming a plurality of different shaped sensing elements in predetermined locations;

FIG. 12a-c depicts a view of a schematic of a method of encasing sensing elements by contact lithography;

FIG. 13A-C depicts examples of sensing elements encased in a polymeric outer layer;

FIG. 14a-c depict a view of a schematic of a method for forming an embodiment of a sensor array with a random array of sensing elements;

FIG. 15a-c depict a view of a schematic of a method for forming an embodiment of a sensor array with an ordered array of sensing elements;

FIG. 16A-F depict several photographs of sensor arrays formed using the methods depicted in FIG. 14 and FIG. 15;

FIG. 17 depicts an embodiment of a device for absorbing extraneous activating light during curing of sensing elements.

### DETAILED DESCRIPTION OF PREFERRED EMBODIMENTS

Herein we describe a system and method for the simultaneous analysis of a fluid containing one or more analytes. The system may be used for either liquid or gaseous fluids. The system may generate patterns that are diagnostic for both individual analytes and mixtures of the analytes. The system, in some embodiments, is made of a combination of sensing elements capable of simultaneously detecting many different kinds of analytes rapidly. An aspect of the system is that the array may be formed using a microfabrication process, thus allowing the system to be manufactured in an inexpensive manner.

### System for Analysis of Analytes

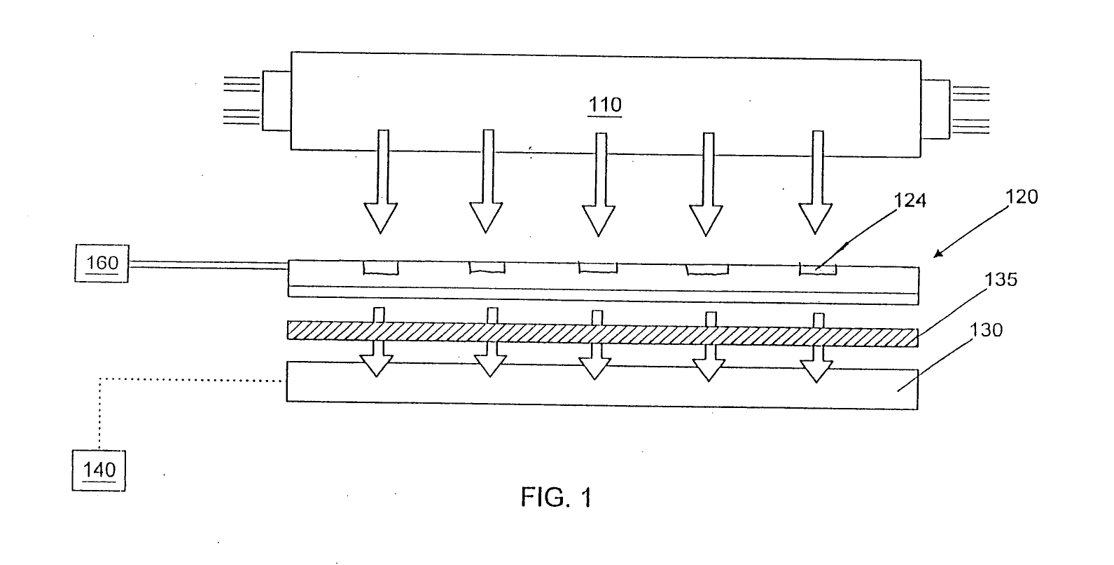

Shown in FIG. 1 is an embodiment of a system for detecting analytes in a fluid. The system, in some embodiments, includes light source 110, sensor 120 and detector 130. Light source 110 may be a white light source or light emitting diodes (LED). In one embodiment, light source 110 may be a blue light emitting diode (LED) for use in systems relying on changes in fluorescence signals. For colorimetric (e.g., absorbance) based systems, a white light source may be used. Sensor 120 is formed of a supporting member which is configured to hold a variety of sensing elements 124. The sensing elements may be configured to produce a detectable signal in the presence of analytes. Each different sensing element may have a unique shape and or size. Detecting device 130 (e.g., a charge-coupled device “CCD”) may be positioned below the sensor to allow for data acquisition. In another embodiment, detecting device 130 may be positioned above the sensor.

Light originating from light source 1 10, in some embodiments, passes through sensor 120 and out through the bottom side of the sensor. The supporting member and the sensing elements together provide an assembly whose optical properties are well matched for spectral analyses. Thus, light modulated by the sensing elements may pass through the sensor and onto proximally spaced detector 130. Evaluation of the optical changes may be completed by visual inspection (e.g., with a microscope) or by use of microprocessor 140 coupled to the detector. For fluorescence measurements, filter 135 may be placed between supporting member 120 and detector 130 to remove the excitation wavelength. Fluid delivery system 160 may be coupled to the supporting member. Fluid delivery system 160 may be configured to introduce samples into and out of the sensor.

The supporting member may be made of any material capable of supporting the sensing elements. The sensing elements may have unique shapes, each of the shapes being associated with one or more analytes. For convenience the sensing elements are depicted have geometrical shapes, however it should be understood that the sensing element may have other shapes. The sensing elements may have a non-spherical shape. Lithographic techniques may be used to fabricate the sensing elements into shapes. The sensing elements may be individually prepared and used to form a sensor. The sensor may be formed by immobilizing the sensing elements in or on a supporting material. Image analysis techniques, as described above, may be used to recognize the shape of the sensing element, and the signal produced in response to the presence or absence of the analyte. Together this information may be used to qualitatively and/or quantitatively identify the analytes present in the fluid sample.

The sensing elements may be produced from a variety of materials. In one embodiment, the sensing elements may be produced from a polymeric material. Examples of polymeric materials, include, but are not limited to, polymers such as Polyethylene glycol hydrogels, poly(ethylene glycol) diacrylate, polydiallylglycol carbonates, cellulosic esters (e.g., cellulose acetate butyrate, cellulose acetate, etc.), polycarbonates, polyphenyl ethers, polyacrylonitrile-butadiene-styrene copolymers, polyvinylchloride, polystyrene, acrylic polymers (e.g., polymethylmethacrylate, etc.), polyester polymers (e.g., polyethylene terephthalate, etc.), polyolefins, (e.g., polyethylene, polypropylene, etc.), fluorocarbon polymers (e.g., polytetrafluoroethylene), polyimides, polyamides, polyurethanes, polyacetals and others known to the art. The sensing element may be produced by polymerization of a monomer composition using either thermal or activating light curing techniques. Alternatively, the sensing elements may be formed by cross-linking a polymeric resin.

In one embodiment, a composition that includes polyethylene glycol (PEG) polymers is used for the fabrication of the sensing elements. Preferably, PEG hydrogel materials may be used. An advantage of using PEG hydrogel materials is that these materials exhibit general resistance to non-specific protein absorption and a wide variety of protein attachment protocols. Furthermore, the porosity of hydrogel materials may be varied to enable the transport of small analyte (e.g., glucose) and large analyte (e.g., protein) molecules for detection.

The sensing elements may be formed using a variety of techniques. Generally, the sensing elements are formed from a composition which is subsequently cured. The curing may be conducted to impart a predefined shape to the sensing element. This shape may be used to identify the specific sensing element. Techniques that may be used to fabricate sensing elements include, but are not limited to, contact lithography, projection lithography, imprint lithography or micromolding based on surface wetting.

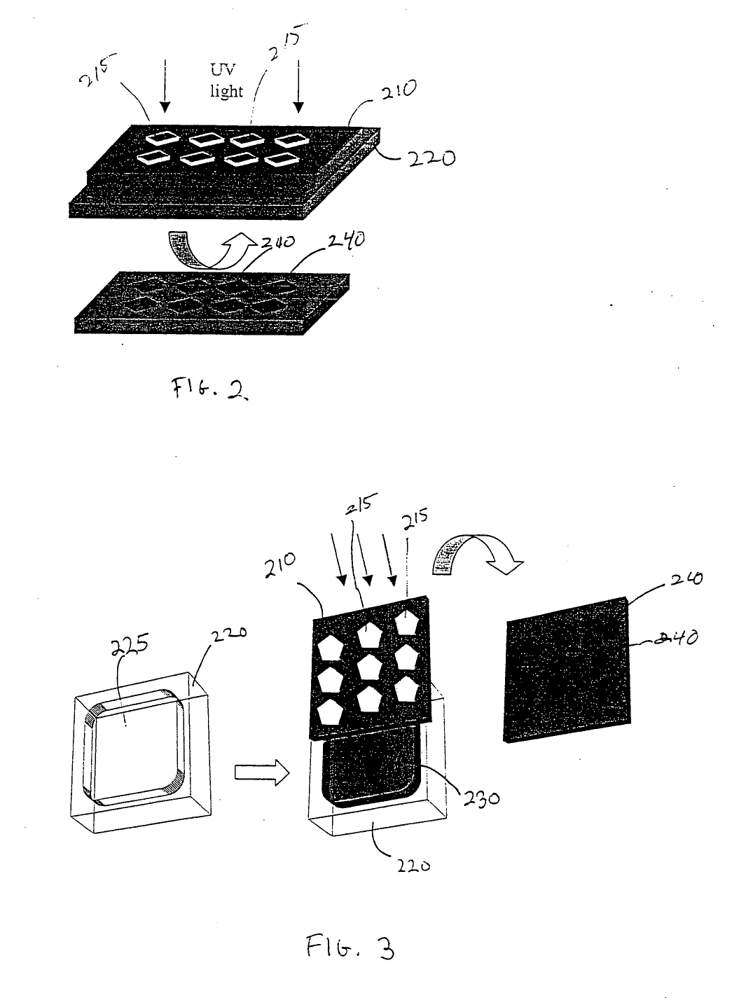

Contact lithography uses photomask templates to cross link liquid monomer materials into sensing elements on an inert substrate (e.g., a glass microscope slide). Referring to FIGS. 2 and 3, mask 210 that includes one or more openings 215 having a predetermined shape is placed on inert substrate 220. Mask 210 may include, but is not limited to, transparencies (such as those used in a laser printer), 35 mm slide film, or patterned chrome on a quartz plate. A secondary mask (not shown) may be placed between mask 210 and composition 230 to protect mask 210. Inert substrate 220 may be, for example, a white Teflon dish. Inert substrate 220 may include cavity 225. Cavity 225 may range from about 0.25-1.0 mm deep. The depth of cavity 225 may control the thickness of the sensing elements. It may be advantageous to use a non-reflective pan instead of a white Teflon dish. The non-reflective pan may reduce UV scattering allowing smaller, higher resolved sensing elements to be formed. Composition 230 used to form the sensing elements may be disposed in cavity 225. Activating light may be applied to the composition disposed within cavity 225 to cure the composition. As used herein “activating light” means light that may affect a chemical change. Activating light may include ultraviolet light (e.g., light having a wavelength between about 300 nm to about 400 nm), actinic light, visible light or infrared light. Generally, any wavelength of light capable of affecting a chemical change may be classified as activating. Chemical changes may be manifested in a number of forms. A chemical change may include, but is not limited to, any chemical reaction that causes a polymerization or a cross-linking reaction to take place. The activating light may be passed through the mask prior to reaching the composition. In this manner the composition may be cured to form the sensing elements. The portions of the composition that are exposed to the activating light may be cured while the unexposed portions of the composition may be substantially uncured. In this manner sensing elements having a shape defined by the openings in mask 210 may be produced.

FIG. 17 shows an embodiment of an apparatus to eliminate nearly all of the reflected UV light. The apparatus is essentially a light trap which may absorb nearly all of the UV after it exposes the uncured sensing elements. The procedure for contact lithography is followed, except a glass substrate may be used in place of the Teflon substrate. The substrate is placed inside the box directly above a reflector. The light may pass through the glass substrate and may reflected into the box, which is painted black. The “black reflector” may be an angled piece of black felt which absorbs nearly all of the light. Any reflected light may be directed towards the black painted walls of the box for further absorption. To reduce reflections from the glass substrate interface, the box may be filled with water, which has an index of refraction (n=1.333) that more closely matches that of the glass (n=1.5). The “light pipe” may be designed to prohibit stray UV from getting into the box.

In addition to the apparatus depicted in FIG. 17, black substrates such as black polystyrene or black carbon filled Teflon can be used to limit reflections.

In one embodiment, depicted in FIG. 2, the composition may include an adhesion promoter that causes the sensing elements to be cross-linked to substrate 220 when the composition is cured. The portions of the composition that are not cross-linked may not adhere to the substrate. After curing is completed, mask 210 may be removed and the uncured portions of the composition may be removed using a suitable solvent. For PEG hydrogel based sensing elements, the uncured composition may be removed with water.

In another embodiment, depicted in FIG. 3, cavity 225 may be coated with a material to reduce the adhesion between the cured composition. After curing of the composition is completed, the uncured composition may be removed and the sensing elements collected. The sensing elements may adhere to the secondary mask and may be collected by scraping them off with, for example, a razor blade.

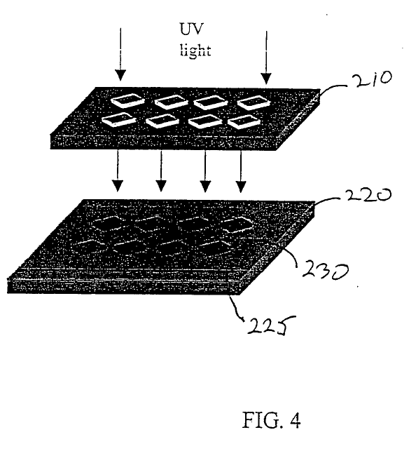

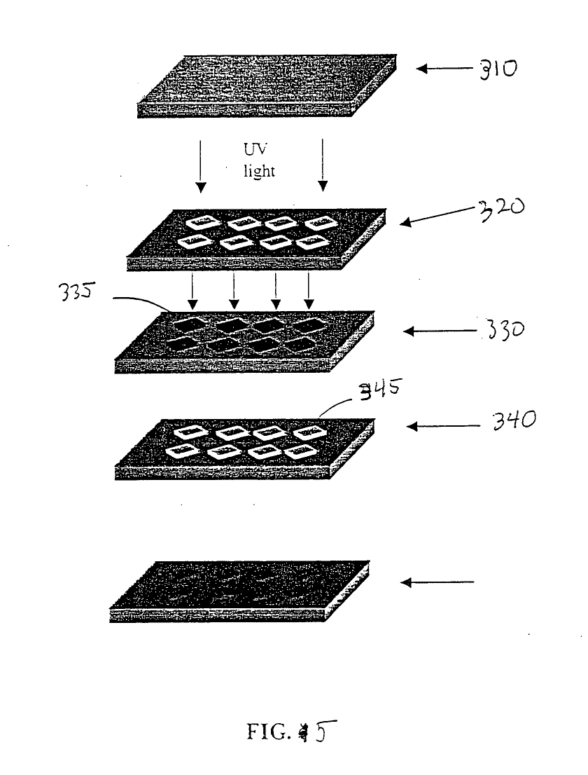

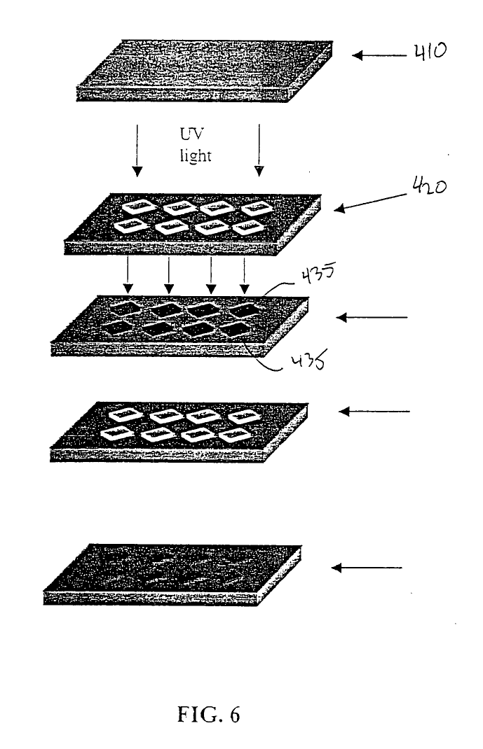

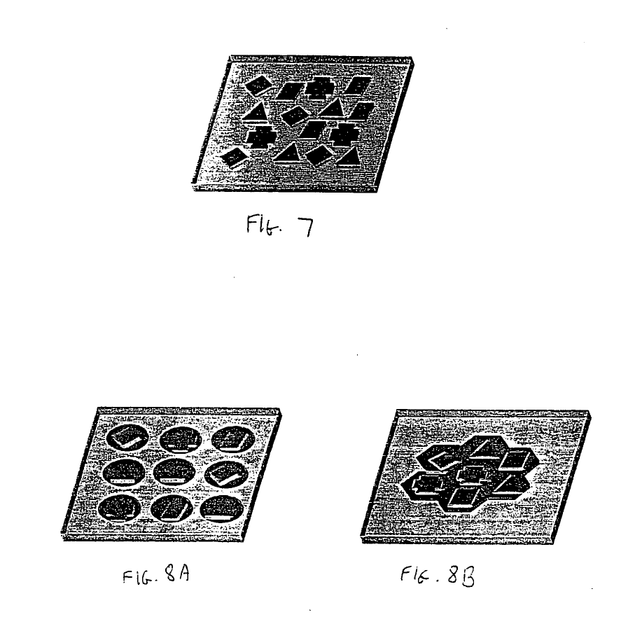

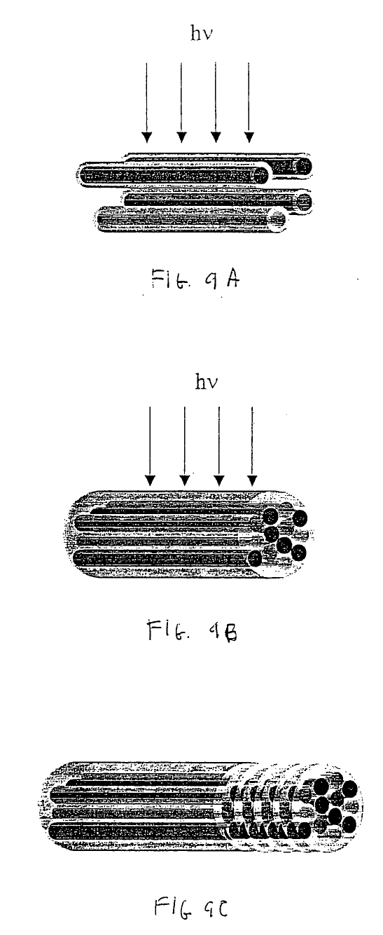

In a similar manner, projection lithography may be used to form the sensing elements. The method of projection lithography is similar to the method described above for contact lithography. Projection lithography differs from contact lithography in that the mask is not in contact with the underlying inert substrate, as depicted in FIGS. 2 and 3. Instead, the mask 210 may be positioned proximate to substrate 220, but not in contact with the substrate, as depicted in FIG. 4. Thus, the patterned light is projected onto composition 230. Substrate 220 may have coated or uncoated cavity 225 configured to receive the composition.

In another embodiment, the sensing elements may be formed using micromolding. Referring to FIG. 5, the micromolding technique may be based on the formation of support 310 having a plurality of wells that may be used to form the sensing elements. The support may, in one embodiment, be coated with a photoresist material (either a dry film or wet photoresist material). The support may be coated with an adhesion promoter prior to coating with the photoresist material to increase the adhesion of the subsequently formed developed photoresist to the support. The photoresist may be developed using photolithography mask 320 and etched. Etching may be performed using dry (e.g., plasma etching) or wet etching techniques. Etching of the photoresist forms a plurality of islands 335 of developed photoresist material on the support. The support may be coated with either a hydrophobic or hydrophilic coating. A hydrophilic coating may be used when the sensing elements are formed from a hydrophobic composition. Alternatively, a hydrophobic coating may be used when the sensing elements are formed from a hydrophilic composition (e.g., a water based composition). After coating the support with the appropriate coating layer, the photoresist islands may be removed 340. Removal of the photoresist islands may leave a plurality of wells 345 disposed within the coating. When the coated substrate is treated with the composition, the composition is attracted to the wells, while being repelled by the coated surface of the support. Thus the composition attains the shape of the “molds” formed in the coating layer.

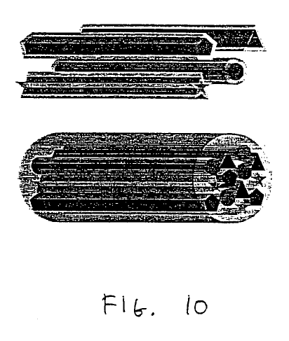

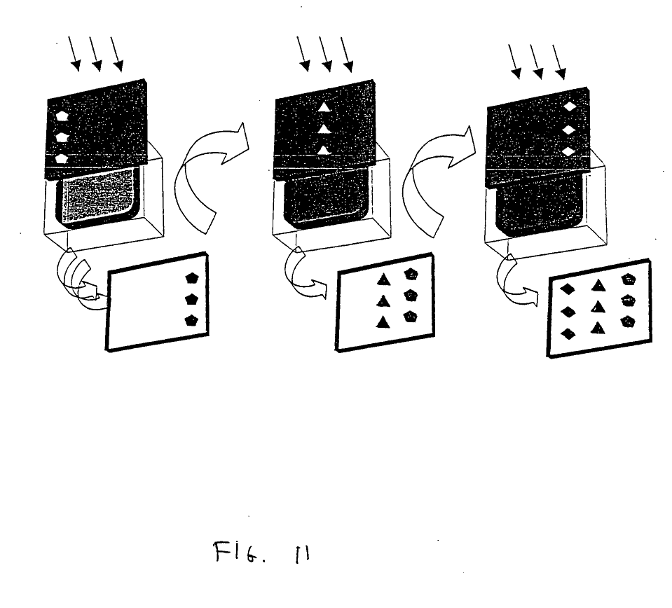

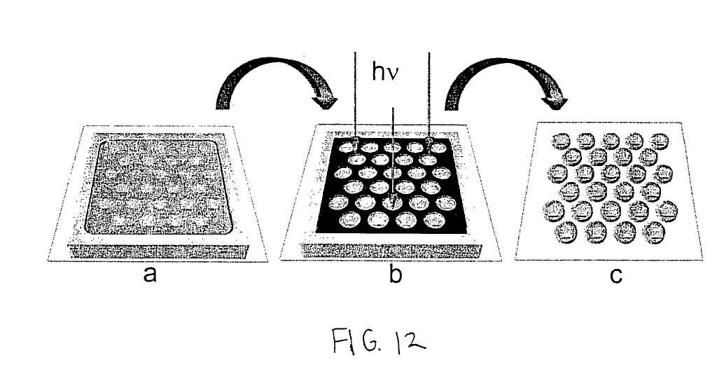

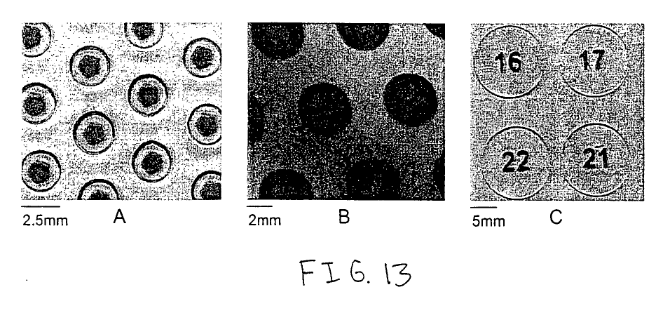

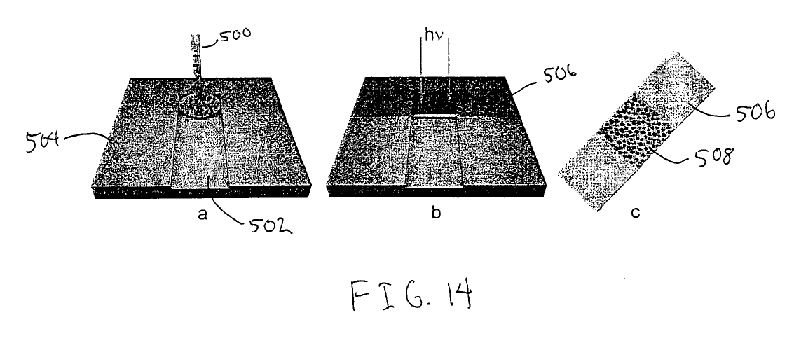

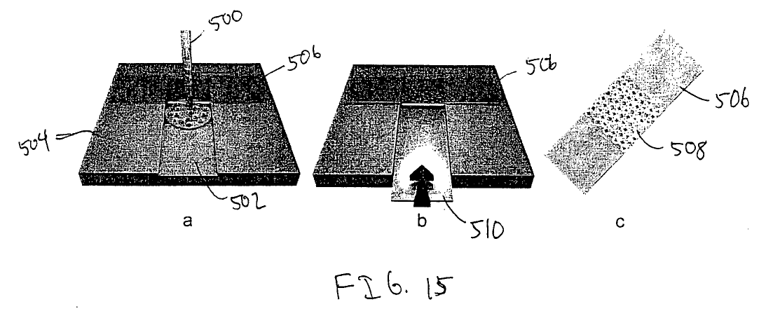

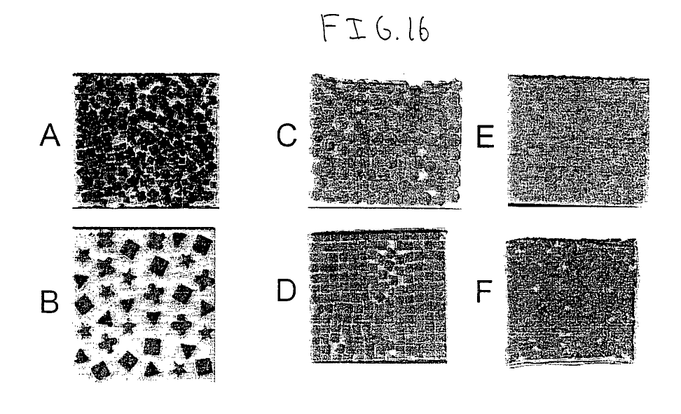

In an alternate method, depicted in FIG. 6, a plurality of molding wells may be formed in a photoresist material. Support 410 may, in one embodiment, be coated with a photoresist material (either a dry film or wet photoresist material). The support may be coated with an adhesion promoter prior to coating with the photoresist material to increase the adhesion of the subsequently formed developed photoresist to the support. The photoresist may be developed using photolithography mask 420 and etched. Etching may be performed using dry (e.g., plasma etching) or wet etching techniques. In contrast to the above-described example, a negative photoresist material may be used. Thus, etching of the photoresist forms a plurality of wells 435 disposed within the undeveloped photoresist material on the support. Wells 435 may be filed with a composition and the composition cured to form sensing elements disposed with wells 435. The photoresist material may be removed to form a plurality of sensing elements disposed on the substrate.

After the sensing elements have been formed, a receptor may be bound to the sensing element. The bound receptor may interact with an analyte to produce a detectable signal. The sensing element may be formed as described above, and the receptors subsequently coupled to the sensing element. Alternatively, the sensing elements may be coupled to a supporting member, as described below, and the receptor may be subsequently coupled to the sensing element.

The sensing elements may be coupled to a supporting member. As described before the sensing element may be coupled to the supporting member during the formation of the sensing elements. In some embodiments, the sensing element may be coupled to a supporting member via crosslinking reactions that occur during formation of the sensing elements. The sensing elements may be coupled to the supporting member such that the sensing elements are disposed on or at an exterior surface of the supporting member.

Alternatively, the supporting member may be formed of a liquid curable composition. The sensing elements may be placed in the liquid curable composition and the composition cured to form the sensor. In this embodiment, the sensing elements are disposed at an interface of the supporting member to allow the sensing elements to interact with the fluid that include the analyte. The sensing elements may be disposed either at the top surface of the supporting member or the bottom surface of the supporting member.

In one embodiment, the liquid composition used to form the supporting member has a density that is less than the density of the sensing elements. When disposed in the liquid composition, the sensing elements will sink to the bottom of the composition. Subsequent curing of the composition will produce a sensor that includes sensing elements disposed at the bottom of the sensor array. Alternatively, the composition may have a density that is greater than the density of the sensing elements. In this situation the sensing elements may float to the surface of the composition. Subsequent curing of the composition will produce a sensor with sensing elements disposed at the top surface of the supporting member.

The orientation of the sensing elements in the supporting member may be random or ordered. In some embodiments, the orientation of the sensing elements may depend on the method of manufacturing used and the material chosen. For example, the choice of materials may allow the sensing elements to be disposed in a self-assembled ordered pattern. Self-assembly forces may be driven by adhesion (sensing element to sensing element or sensing element to a solid surface), capillary bonds, gravity and surface tension.

FIG. 7 depicts sensing elements disposed within a support member. The sensing elements may be randomly dispersed within the support member, as shown. Alternatively, the sensing elements may be in an ordered array as depicted in FIGS. 8A and 8B. In one embodiment, the sensor may be made from a liquid composition that is cured to form the supporting member. The supporting member may be formed from a mold that has a plurality of wells disposed in an ordered array. The liquid composition may be added to the mold such that the wells are at least partially filled with the liquid composition. Sensing elements may be added to the liquid composition and the sensing elements may sink into the wells. The liquid composition is cured and the formed sensor removed from the molds. The sensing elements will be disposed within the sensor in an ordered array complementary to the pattern of wells in the mold. This method may be used to make arrays, as depicted in FIG. 8A or predetermined patterns of sensing elements.

In some embodiments, a sensor array may be formed with the sensing elements in a random order. Sensing elements may be mixed together in a polymerizable solution. The solution of sensing elements may be drawn into pipet 500 or any such measured dispensing device. Pipet 500 may then dispense the sensing element solution into cavity 502 in tray 504 depicted in FIG. 14a. The sensing elements may have a higher density than the polymerizable solution and therefore sink to the bottom of cavity 502. Cavity 502 may be cut to a depth slightly greater than the height of the sensing elements. For example, if the sensing elements are about 0.5 mm in height, cavity 502 may be about 0.64 mm in depth. A depth that is greater than the height of the sensing elements and substantially less than twice the height of the sensing elements may inhibit the sensing elements from stacking one on top of another while allowing the sensing elements to move around in cavity 502. Slide 506 may be positioned over the solution of sensing elements in cavity 502. The solution of sensing elements may be exposed to activating light inducing polymerization of the solution of sensing elements, as shown in FIG. 14b. Polymerized sensor array 508 may adhere to slide 506, advantageously providing a convenient substrate for sensor array 508 shown in FIG. 14c.

In another embodiment, a sensor array may be formed with the sensing elements in a close packed array, as shown in FIG. 15a-c. Slide 506 may be anchored or coupled to tray 504 where slide 506 may be positioned over a portion of cavity 502. A polymerizable solution of sensing elements may be dispensed with pipet 500 into cavity 502 next to slide 506, shown in FIG. 15a. Device 510, such as a portion of a silicon wafer, may be employed to push/position the sensing elements in a close-packed array in the opening created by cavity 502 and slide 506, shown in FIG. 15b. Polymerization of the solution of sensing elements may be induced with activating light forming an ordered array of sensing elements.

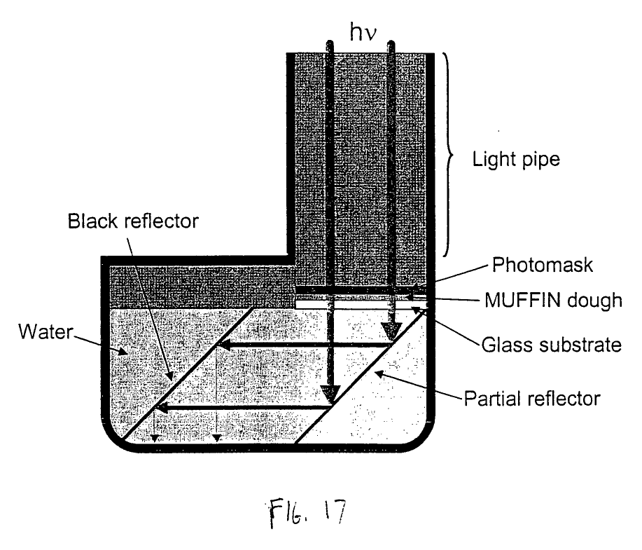

FIG. 16A-F depict several photographs of sensor arrays formed using the methods described herein. FIG. 16A depicts an array of cross, square, and triangle shaped sensing elements formed using the random arraying approach. FIG. 16B depicts an array of encapsulated sensing elements formed using the close packed arraying approach. FIG. 16C-F depict how circles (C), squares (D), hexagons (E), and triangles (F) pack using the close packed arraying method.

In another embodiment, the sensing elements may be formed in elongated form. FIG. 9A depicts a plurality of elongated sensing elements. Each of the sensing elements may include a receptor that interacts with the analyte. The elongated sensing elements may be formed by placing a liquid composition in an elongated mold and curing the liquid composition within the mold. In some embodiments, the elongated sensing elements may be only partially crosslinked. This may allow a thin film of uncrosslinked material to remain along the inside surface of the mold. This may allow the elongated sensing elements to be more easily removed. The individual elongated sensing elements may be placed in a larger tube containing a curable composition, FIG. 9B. The tube may be cured such that the composition is substantially crosslinked. In this manner the curable composition may be converted to a support member for the elongated sensing elements. The elongated sensing elements may be cut, as depicted in FIG. 9C into smaller sensors. The production of sensors in this manner may allow the rapid production of multiple sensors.

This method of using elongated sensing elements to create multiple sensors may be expanded by using different shaped tubes for the sensors, as depicted in FIG. 10. When these sensors are combined into a random array the shapes may be used to determine the particular sensor.

Sensing elements may also be formed with direct addressibility as depicted in FIG. 11. The method may use multiple lithography steps to produce a variety of different shaped sensing elements, as depicted in FIG. 11. Alternatively, a single mask having a variety of different patterns may be used to produce different shaped elements. Wells may be used to organize the sensing elements in ordered arrays or predetermined patterns.

In some embodiments, the sensing elements may be encased in a polymeric outer layer. The polymeric outer layer may be concentric. Contact lithography as described herein may be used to encapsulate the sensing elements, as depicted in FIG. 12. The sensing elements adhering to the secondary transparent mask may be immersed in a second polymerizable composition as depicted in FIG. 12a. A mask with concentric shapes may be placed over the sensing elements in the composition, shown in FIG. 12b. Activating light may be used to promote polymerization of the composition. Excess composition may then be washed away with the appropriate solvent with the encased sensing elements adhering to the mask (FIG. 12c). Several advantages to encasing the sensing elements include protecting the distinctive shape of the sensing elements when they are packed closely together. Other advantages includes increasing the number of distinctive shapes available, including a large range of numbers for example. The second polymerizable composition which encases the sensing elements may not be the same as the composition used to form the sensing elements allowing different characteristics to be imparted to the sensing elements such as structural integrity. The shape of the encasing composition may affect how the sensing elements are arrayed when packed together, circular sensors may arrange in a hexagonal close packed array and squares may arrange in a tight grid. Examples of sensing elements encased in an outer layer are depicted in FIGS. 13A-C.

### Sensing Elements

A sensing element, in some embodiments, possess both the ability to bind the analyte of interest and to create a modulated signal. The sensing element may include receptor molecules which posses the ability to bind the analyte of interest and to create a modulated signal. Alternatively, the sensing element may include receptor molecules and indicators. The receptor molecule may posses the ability to bind to an analyte of interest. Upon binding the analyte of interest, the receptor molecule may cause the indicator molecule to produce the modulated signal. The receptor molecules may be naturally occurring or synthetic receptors formed by rational design or combinatorial methods. Some examples of natural receptors include, but are not limited to, DNA, RNA, proteins, enzymes, oligopeptides, antigens, and antibodies. Either natural or synthetic receptors may be chosen for their ability to bind to the analyte molecules in a specific manner. The forces which drive association/recognition between molecules include the hydrophobic effect, anion-cation attraction, and hydrogen bonding. The relative strengths of these forces depend upon factors such as the solvent dielectric properties, the shape of the host molecule, and how it complements the guest. Upon host-guest association, attractive interactions occur and the molecules stick together. The most widely used analogy for this chemical interaction is that of a “lock and key.” The fit of the key molecule (the guest) into the lock (the host) is a molecular recognition event.

A naturally occurring or synthetic receptor may be bound to a polymeric resin having a predetermined shape in order to create the sensing element. In one embodiment, the material used to form the polymeric resin is compatible with the solvent in which the analyte is dissolved. For example, PEG hydrogel resins will swell within polar solvents, but does not significantly swell within non-polar solvents. Thus, PEG-hydrogel resins may be used for the analysis of analytes within polar solvents.

In an embodiment, living bacterial cells may be used as a receptor in a sensing element. One example might be E. Coli cells engineered to express green fluorescence protein (GFP) when induced with arabinose. However, to protect the cells from free radical polymerization processes, the cells may be first incorporated into agarose. The agarose may then be ground into fine fragments and mixed into a polymerizable composition.

The sensing element, in one embodiment, is capable of both binding the analyte(s) of interest and creating a detectable signal. In one embodiment, the sensing element will create an optical signal when bound to an analyte of interest. In one embodiment, a detectable signal may be caused by the altering of the physical properties of an indicator ligand bound to the receptor or the polymeric resin. In one embodiment, two different indicators are attached to a receptor or the polymeric resin. When an analyte is captured by the receptor, the physical distance between the two indicators may be altered such that a change in the spectroscopic properties of the indicators is produced. A variety of fluorescent and phosphorescent indicators may be used for this sensing scheme. This process, known as Forster energy transfer, is extremely sensitive to small changes in the distance between the indicator molecules.

Alternatively, the first and second fluorescent indicators may initially be positioned such that short wavelength excitation, may cause fluorescence of both the first and second fluorescent indicators, as described above. After binding of analyte to the receptor, a structural change in the receptor molecule may cause the first and second fluorescent indicators to move further apart. This change in intermolecular distance may inhibit the transfer of fluorescent energy from the first indicator to the second fluorescent indicator. This change in the transfer of energy may be measured by either a drop in energy of the fluorescence of the second indicator molecule, or the detection of increased fluorescence by the first indicator molecule.

In another embodiment, an indicator ligand may be preloaded onto the receptor. An analyte may then displace the indicator ligand to produce a change in the spectroscopic properties of the sensing elements. In this case, the initial background absorbance is relatively large and decreases when the analyte is present. The indicator ligand, in one embodiment, has a variety of spectroscopic properties which may be measured. These spectroscopic properties include, but are not limited to, ultraviolet absorption, visible absorption, infrared absorption, fluorescence, and magnetic resonance. In one embodiment, the indicator is a dye having either a strong fluorescence, a strong ultraviolet absorption, a strong visible absorption, or a combination of these physical properties. When the indicator is mixed with the receptor, the receptor and indicator interact with each other such that the above mentioned spectroscopic properties of the indicator, as well as other spectroscopic properties may be altered. The nature of this interaction may be a binding interaction, wherein the indicator and receptor are attracted to each other with a sufficient force to allow the newly formed receptor-indicator complex to function as a single unit. The binding of the indicator and receptor to each other may take the form of a covalent bond, an ionic bond, a hydrogen bond, a van der Waals interaction, or a combination of these bonds.

The indicator may be chosen such that the binding strength of the indicator to the receptor is less than the binding strength of the analyte to the receptor. Thus, in the presence of an analyte, the binding of the indicator with the receptor may be disrupted, releasing the indicator from the receptor. When released, the physical properties of the indicator may be altered from those it exhibited when bound to the receptor. The indicator may revert back to its original structure, thus regaining its original physical properties. For example, if a fluorescent indicator is attached to a sensing element that includes a receptor, the fluorescence of the sensing element may be strong before treatment with an analyte containing fluid. When the analyte interacts with the sensing element, the fluorescent indicator may be released. Release of the indicator may cause a decrease in the fluorescence of the sensing element, since the sensing element now has less indicator molecules associated with it.

In an embodiment, the analyte molecules in the fluid may be pretreated with an indicator ligand. Pretreatment may involve covalent attachment of an indicator ligand to the analyte molecule. After the indicator has been attached to the analyte, the fluid may be passed over the sensing elements. Interaction of the receptors on the sensing element s with the analytes may remove the analytes from the solution. Since the analytes include an indicator, the spectroscopic properties of the indicator may be passed onto the sensing element. By analyzing the physical properties of the sensing element s after passage of an analyte stream, the presence and concentration of an analyte may be determined.

For example, the analytes within a fluid may be derivatized with a fluorescent tag before introducing the stream to the sensing elements. As analyte molecules are adsorbed by the sensing element, the fluorescence of the sensing element may increase. The presence of a fluorescent signal may be used to determine the presence of a specific analyte. Additionally, the strength of the fluorescence may be used to determine the amount of analyte within the stream.

### Receptors

A variety of natural and synthetic receptors may be used. The synthetic receptors may come from a variety of classes including, but not limited to, polynucleotides (e.g., aptamers), peptides (e.g., enzymes and antibodies), synthetic receptors, polymeric unnatural biopolymers (e.g., polythioureas, polyguanidiniums), and imprinted polymers. Natural based synthetic receptors include receptors which are structurally similar to naturally occurring molecules. Polynucleotides are relatively small fragments of DNA which may be derived by sequentially building the DNA sequence. Peptides may be synthesized from amino acids. Unnatural biopolymers are chemical structure which are based on natural biopolymers, but which are built from unnatural linking units. Unnatural biopolymers such as polythioureas and polyguanidiniums may be synthesized from diamines (i.e., compounds which include at least two amine functional groups). These molecules are structurally similar to naturally occurring receptors, (e.g., peptides). Some diamines may, in turn, be synthesized from amino acids. The use of amino acids as the building blocks for these compounds allow a wide variety of molecular recognition units to be devised. For example, the twenty natural amino acids have side chains that possess hydrophobic residues, cationic and anionic residues, as well as hydrogen bonding groups. These side chains may provide a good chemical match to bind a large number of targets, from small molecules to large oligosaccharides.

Techniques for the building of DNA fragments and polypeptide fragments on a polymer particle are well known. Techniques for the immobilization of naturally occurring antibodies and enzymes on a polymeric resin are also well known.

### EXAMPLES

### 1. Sensing Element Composition

The sensing elements are composed of PEG hydrogels that are cast in a liquid form and cured. The amount of water mixed with the hydrogel determines the level of swelling that may occur in the presence of water as well as the mechanical properties of the muffin. The composition includes:

| | |
|---|---|
| PEG-20k-bisacrylate | 5% |
| PEG-300-monoacrylate | 45% |
| Phosphate buffer (PBS) | 38% |
| Darocure 1173 | 2% |
| Fluorescent Enzyme | 10% |

### PBS Buffer Composition:

| | |
|---|---|
| KH₂PO₄ | 0.144 g/l |
| NaCl | 9.00 g/l |
| Na₂HPB₄*7H₂O | 0.795 g/l |

### 2. Sensing Enzymes

The following enzymes were coupled to the sensing elements as receptors for the indicated analyte:

glucose oxidase (10 mg/ml)—glucose (308 mg/ml)

urea oxidase (10 mg/ml)—urea (10 mg/ml)

acetylcholinesterase (5 mg/ml)—acetylcholine

All enzymes have a fluorescent tag SNAFL that is excited 514 nm and emits light in a range from 525 nm to 625 nm. In general, the emitted fluorescent signal from SNAFL attached to the enzyme increases or decreases (in green or red) as a function of pH. The glucose oxidase and acetylcholinesterase react with their respective materials to form an acid that shifts the fluorescent intensity deeper into the green. The ureaoxidase reacts with urea to form a base that moves the signal from green to a strong red. This color shift to the red appears stronger than the other sensors.

### 3. Formation of Sensing Elements

The enzyme is currently added to the liquid sensing element composition of Example 1 before it is cross-linked. The curing conditions/free radical generator must require low exposure dose to cure the system while preventing the enzyme from losing its activity. Initial experiments reveal that 100 mJ/cm² is needed to cure a 20 mils thick muffin using Durocure 1173. The ultraviolet light source has an output estimated at 200 mW/cm² thus requiring around ⅛ of a second for exposure to cure.

### 4. Glucose Sensing Experiment

Sensing Element Production:
3.5 ml of PEG matrix was added to 0.35 ml of glucose oxidase
Sensors were used at a concentration of 100 microliter of the enzyme solution per ml of PEG.
PEG/enzyme matrix was pipette into the Teflon pan with 1 mls of depth
The template curing method was used to cure (1 sec) shaped muffins directly to a microscope slide that had a transparency mask attached to the other side

Analysis of Sensing Elements (Fluorescent Microscope—Gray Scale Image Analysis)
Standard pH solutions were used to determine the dye activity
Muffins on glass were immersed in 2 mmol PBS to leach out 1 molar PBS
Muffins were then immersed in standard pH solutions for 5 minutes
Basic (pH 11.7) bright fluorescence in red
Neutral (DI water pH 6.8) bright fluorescence in green

Glucose Sensing
Muffins were immersed in 2 mmole PBS solution to leach-out 0.1 molar PBS
Glucose (308 mg/ml) was dripped on the microscope slide containing the muffins
The enzyme should produce more acidic conditions that drive the fluorescence from red to green: results: the red signal decreased, but the green signal also seemed to decrease relative to the initial fluorescence signal. However, the liquid droplets wetting the muffins on the microscope slides could be affecting the optical properties of the signal being received by the fluorescence microscope.

### 5. Urea Sensing Experiment

Urea was added to the sensing elements on a glass microscope slide using the same protocol as described above for the glucose experiment.
Ureaoxidase reacts with urea to form a base that strongly drove the fluorescent signal from the green to red;
This experiment successfully demonstrated a strong red signal that could easily be identified by the shape of the sensing element.

### 6. pH Sensing Experiment

A demonstration of chemical detection was accomplished by making pH sensitive, concentric sensing elements. Three pH sensitive dyes were encapsulated into stars (methyl purple), triangles (congo red) and squares (phenol red). The inner sensing elements were made from a composition including 48-wt % pH dye in water, 50-wt % PEG-575-diacrylate and 2-wt % Darocur 1173. The composition for the immobilizing matrix consisted of 73-wt % PEG-575-diacrylate, 25-wt % deionized water, and 2-wt % Darocur 1173.

The array containing the pH sensors was placed in both acidic (1M HCl pH˜1) and basic (0.26N tetramethylammonium hydroxide pH>10) solutions. The sensing element dyes changed color successfully sensing pH changes.

### 7. DNA Sensing Experiment

Sensing elements were made with both encapsulated and chemically bound single stranded DNA 18-mers for complementary hybridization sensing. Oligonucleotides were synthesized using standard methods for automated DNA synthesis with nucleoside phosphoramidites. The oligonucleotides were synthesized on a 0.2 μmol scale, using an Expedite Nucleic Acid Synthesis System. A 3′-rhodamine tagged oligonucleotide [AATTCAATAAGGTGGTAT(R)] was encapsulated in a cross-shaped sensing element. A 3′-rhodamine tagged oligonucleotide with a 5′-acrylate functional group [(Acry)ATACCAGCTTATTCAATT(R)] was chemically incorporated into pentagon shaped sensing elements via copolymerization. The sensing elements were made as described herein except the dye solution was replaced with 12 μM DNA.

The derivatized sensing elements were washed with buffer multiple times. The pentagon shaped sensing elements which incorporated the covalently bound 3′-rhodamine, 5′-acrylate DNA oligomer displayed a bright fluorescent signal. The cross-shaped sensing elements which contained the encapsulated 3-rhodamine tagged oligonucleotide showed a much weaker signal. The encapsulated DNA diffused out of the sensing element during rinsing, while the covalently bound DNA was retained. The center of the cross still showed a weak signal, which can be attributed to the small amount of encapsulated DNA which had not yet diffused from the center of the sensing element. Clearly, unbound 18-mer DNA is capable of diffusing out of the sensing elements.

To test the hybridization capability of covalently bound DNA, a 5′-acrylated oligonucleotide [(Acry)ATACCAGCTTATTCAATT] sensor was copolymerized into triangular sensing elements. A 3′-fluoresceinylated oligonucleotide of complementary sequence [AATTGAATAAGCTGGTAT(F)] was used as a target for hybridization. The triangular sensing elements were soaked in 10 μL of a 50 μM solution of the complimentary DNA oligonucleotide (0.5 nmol) and rinsed. A bright signal was observed, indicating that the 5′-acrylated oligonucleotide had hybridized with the compliment oligonucleotide within the triangular sensing elements. Square sensing elements containing no DNA sensors were also soaked over night in 10 μL of a 50 μM solution of the 3′-fluoresceinylated oligonucleotide (0.5 nmol). The square sensing elements demonstrated that there is a minimal fluorescent signal due to non-specific adsorption of oligonucleotides.

### 8. Cell Based Sensing Experiment

Detection of digoxigenin was demonstrated using E. coli displaying single chain antibody fragments (scFv) specific for the cardiac glycoside digoxin on their surface. Digoxin specific E. coli in PBS buffer were encapsulated in a square shaped sensing element. As a control, E. coli displaying scFv specific for the herbicide atrazine were encapsulated in a triangle shaped sensing element. The pre-polymer mixture for both sensing elements contained 25-wt % PEG 575, 1-wt % Darocur 1173, 68-wt % 0.05M NaOH, and a 6-wt % mixture of E. coli in PBS. The optical density of the cell mixture was approximately 100 optical density (O.D.) at 600 nm. A second control with no cells was cast as a circle shaped sensing element.

The sensing elements were incubated for 1 hour in a PBS solution containing 100 nM BODIPY-digoxigenin probe, and 15 μM of propidium iodide (PI). The PI stains dead cells by fluorescing red. The sensing elements were then rinsed in a 0.05 mM solution of a mild non-ionic detergent, NP-40, in PBS to remove any unbound probe. Finally, the sensing elements were imaged on a fluorescent microscope at 4× magnification. The results show that only the sensing element containing the cells with the digoxin antibody fragments on their surface bound the probe.

### 9. Muffin Immobilization Experiment

The ureaoxidase sensors (as described in Example 5) were removed from the glass slide and placed into a Teflon template that contained the PEG composition without any enzymes. The whole template was exposed for 1 second to cure the sensors into a thin film of PEG matrix. The sensing elements immobilized in the non-active PEG matrix still revealed their fluorescent shape recognition. The fluorescent signal was reduced relative to the original sensing elements. This may be due to the double exposure or an increase in the thickness of the matrix that provides a longer path length for detection. However, the experiment still successfully demonstrated shape recognition within an immobilized matrix.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed:

1. A device, comprising a plurality of sensing elements randomly dispersed on a supporting member, wherein at least a portion of which comprises sensing elements of different, non-spherical shapes, and wherein each different sensing element shape is associated with reactivity to a different analyte.

2. The device of claim 1, wherein said sensing elements comprise polymeric material.

3. The device of claim 2, wherein the polymeric material of the sensing elements comprises a polymeric resin.

4. The device of claim 3, wherein the polymeric resin comprises a polyethylene glycol hydrogel resin.

5. The device of claim 4, wherein the polyethylene glycol hydrogel resin is cast in a liquid form and cured.

6. The device of claim 1, wherein each sensing element has a different receptor.

7. The device of claim 6, wherein the receptor is configured to produce a signal when the sensing element interacts with the analyte during use.

8. The device of claim 1, wherein the non-spherical shape is selected from the group consisting of crosses, squares and triangles.

9. A device, comprising a plurality of sensing elements randomly dispersed and capable of movement on a supporting member, wherein at least a portion of which comprises sensing elements of different, non-spherical shapes, and wherein each different sensing element shape is associated with reactivity to a different analyte.

10. The device of claim 9, wherein said sensing elements comprise polymeric material.

11. The device of claim 10, wherein the polymeric material of the sensing elements comprises a polymeric resin.

12. The device of claim 11, wherein the polymeric resin comprises a polyethylene glycol hydrogel resin.

13. The device of claim 12, wherein the polyethylene glycol hydrogel resin is cast in a liquid form and cured.

14. The device of claim 9, wherein each sensing element has a different receptor.

15. The device of claim 14, wherein the receptor is configured to produce a signal when the sensing element interacts with the analyte during use.

16. The device of claim 9, wherein the non-spherical shape is selected from the group consisting of crosses, squares and triangles.

17. The device of claim 9, further comprising a cavity within which said sensing elements can move around.

18. A method, comprising:
a) providing a supporting member and a plurality of sensing elements, wherein each sensing element has a different, non-spherical shape, and wherein each sensing element is capable of reacting with a different analyte; and
b) randomly dispersing said sensing elements on said supporting member.

19. The method of claim 18, wherein said supporting member comprises a cavity within which said sensing elements can move around.

20. The method of claim 18, wherein said sensing elements are suspended in a liquid prior to step b).

21. The method of claim 18, further comprising after step b), contacting said sensing elements with a plurality of different analytes.

22. A method, comprising:
a) providing a supporting member comprising a cavity and a plurality of sensing elements, wherein each sensing element has a different, non-spherical shape, and wherein each sensing element is capable of reacting with a different analyte; and
b) randomly dispersing said sensing elements into said cavity of said supporting member under conditions wherein said sensing elements can move around within said cavity.

23. The method of claim 22, wherein said sensing elements are suspended in a liquid prior to step b).

24. The method of claim 22, further comprising after step b), contacting said sensing elements with a plurality of different analytes.

25. The method of claim 24, wherein a spectroscopic change is caused by the interaction of analyte with the sensing elements.


SEQUENCE LISTING (4 sequences)

SEQ ID NO: 1 - DNA, 18 bp, Artificial Sequence
  Synthetic
aattcaataaggtggtat

SEQ ID NO: 2 - DNA, 18 bp, Artificial Sequence
  Synthetic
ataccagcttattcaatt

SEQ ID NO: 3 - DNA, 18 bp, Artificial Sequence
  Synthetic
ataccagcttattcaatt

SEQ ID NO: 4 - DNA, 18 bp, Artificial Sequence
  Synthetic
aattgaataagctggtat